(19) United States
(12) Patent Application Publication
(10) Pub. No.: US 2009/0264775 A1
(43) Pub. Date: Oct. 22, 2009

(54) BLOOD VESSEL AGEING INDEX MEASURING AND ANALYZING SYSTEM, AND MEASURING AND ANALYZING METHOD THEREOF

(75) Inventors: Hsien-Tsai Wu, Hualien (TW); Chun-Ho Lee, Yilan County (TW); Tsang-Chih Wu, Taipei County (TW); An-Bang Liu, Hualian (TW)

Correspondence Address: POTTER ANDERSON & CORROON LLP;ATTN: JANET E. REED, PH.D., P.O. BOX 951, WILMINGTON, DE, 19899-0951, US

(73) Assignee: Hsien-Tsai Wu

(21) Appl. No.: 12/416,526

(22) Filed: Apr. 1, 2009

(30) Foreign Application Priority Data
Apr. 18, 2008 (TW) 097114065

(51) Int. Cl. A61B 5/02

(52) U.S. Cl. 600/500

(57) ABSTRACT

A blood vessel ageing index measuring and analyzing system includes a pressure applying unit, a measuring unit, a front end signal processing unit, and a back end signal analyzing unit. The pressure applying unit is configured to apply external pressure to a limb of a subject, or to release the limb of the subject from the external pressure. The measuring unit is configured to output a standard radial artery pulse signal and a comparative radial artery pulse signal. The front end signal processing unit is configured to process the standard and comparative radial artery pulse signals to generate a digital standard radial artery pulse signal and a digital comparative radial artery pulse signal. The back end signal analyzing unit is configured to obtain a blood vessel ageing index according to the digital standard and comparative radial artery pulse signals.

### CROSS-REFERENCE TO RELATED APPLICATION

This application claims priority of Taiwanese Application No. 097114065, filed on Apr. 18, 2008.

### BACKGROUND OF THE INVENTION

1. Field of the Invention

The present invention relates to a non-invasive system and method for measuring and analyzing blood vessel ageing indices, more particularly to a blood vessel ageing index measuring and analyzing system and a method thereof that can be used in a hospital or for personal health care for measuring the endothelial cell function of blood vessels so as to evaluate early stages of coronary atherosclerosis and capillary angiosclerosis.

2. Description of the Related Art

Currently, there are many researches proving that there exists a high degree of relation between coronary atherosclerosis and risk factors of various cardiovascular diseases. At present, Pulse Wave Velocity (PWV) is the most general non-invasive method for detecting coronary atherosclerosis. While there are many researches that have verified the relation between PWV and coronary atherosclerosis, PWV can only be used for determining the presence of coronary atherosclerosis, and cannot be used for evaluating diseases related to coronary atherosclerosis at an early stage. Therefore, how to detect coronary atherosclerosis at an early stage and analyze blood vessel ageing through a non-invasive scheme have now become important objects of research.

Many medical documents have stated that evaluation of the endothelial cell function of blood vessels can serve as a reference index for the early stage of coronary atherosclerosis and blood vessel ageing. The inside walls of arteries and capillaries have a layer of endothelial cells. Healthy endothelial cells have two functions: maintenance of tension of blood vessels and coagulant ability in the short term, and activation of smooth muscle cells and assistance in growth of cells in the long term. The endothelial cells can generate nitric oxide (NO), a gaseous neurotransmitter responsible for signal transmission in living organisms. After being generated by the endothelial cells, nitric oxide is diffused to the smooth muscle cells of the blood vessels to inhibit contraction of vascular smooth muscles. Because the smooth muscles control contraction of the blood vessels, the blood vessels dilate when nitric oxide restricts the contraction of the smooth muscles. Therefore, it can be said that the amount of nitric oxide directly affects the extent of dilatation of the arteries and capillaries.

When the endothelial cells are dysfunctional or impaired, the blood vessels are unable to dilate normally. Moreover, continuous impairment of the endothelial cells results in lipid buildup that is one of the causes of initial formation of coronary atherosclerosis and blood vessel ageing. Because endothelial dysfunction is an initial symptom of coronary atherosclerosis and blood vessel ageing, it will be a significant advancement in early diagnosis of diseases related to coronary atherosclerosis and blood vessel ageing if there is a non-invasive method for screening the endothelial cell function of blood vessels.

U.S. Pat. No. 7,077,809 discloses a non-invasive vasodilatation index measuring and analyzing system for evaluating the endothelial cell function. The vasodilatation index measuring and analyzing system measures digit (i.e., fingers and toes) pulse signals of a subject by emitting and receiving an optical signal (i.e., infrared light) through digits of the subject. Then, based on the principle of vasodilatation index, an external pressure larger than a systolic pressure of the subject is applied to the subject's wrist adjacent to the proximal end of the digit to occlude the flow of blood to the tip portion of the digit. After release of the external pressure, an increase in the amount of blood flow results, which is called reactive hyperemia (RH). During this period, an area below the pulse waveform of the digit is calculated. It is found that the pulse area varies with time. This implies that the pulse area variation represents a change in the volume of blood in the blood vessel (vascular volume change). The relationship between the pulse area and the vascular blood volume can thus be expressed as a vasodilatation index (set forth hereunder), which directly reflects the degree of vasodilatation due to reactive hyperemia. The regulating function of endothelial cells can thus be measured to serve as a basis for evaluating atherosclerosis.

Index of Vasodilatation Due to Reactive Hyperemia:

[(Comparative pulse area−standard pulse area)/standard pulse area]×100% (1)

The vasodilatation index measuring and analyzing system according to U.S. Pat. No. 7,077,809 can be used to obtain the vasodilatation index easily for determining the endothelial cell function. However, arterioles of tip portions of fingers are capable of automatically adjusting the flow of blood so as to result in variations of the amount of blood flow. Therefore, there are uncontrollable factors that can cause changes in the pulse signals. Further, a disposed angle of a detector for the optical signal can also result in an error. These may affect accuracy of the measurement result of the vasodilatation index.

### SUMMARY OF THE INVENTION

Therefore, an object of the present invention is to provide a blood vessel ageing index measuring and analyzing system.

Accordingly, a blood vessel ageing index measuring and analyzing system of the present invention comprises a pressure applying unit, a measuring unit, a front end signal processing unit coupled to the measuring unit, and a back end signal analyzing unit coupled to the front end signal processing unit. The pressure applying unit is operable in a pressure-applying mode, where the pressure applying unit is configured to apply external pressure to a limb of a subject, or a pressure-releasing mode, where the pressure applying unit is configured to release the limb of the subject from the external pressure. The measuring unit is associated operatively with the pressure applying unit and is configured to output a standard radial artery pulse signal based upon a pulse of the subject that was measured prior to operation of the pressure applying unit in the pressure-applying mode, and to output a comparative radial artery pulse signal based upon a pulse of the subject that was measured after operation of the pressure applying unit in the pressure-releasing mode. The front end signal processing unit is configured to perform filter processing upon the standard radial artery pulse signal and the comparative radial artery pulse signal outputted by the measuring unit to obtain processed standard and comparative radial artery pulse signals, and is further configured to digitize the processed standard and comparative radial artery pulse signals to generate a digital standard radial artery pulse signal and a digital comparative radial artery pulse signal. The back end signal analyzing unit is configured so as to determine an average value of a standard pulse area with reference to the digital standard radial artery pulse signal, and so as to determine a maximum value of a comparative pulse area with reference to the digital comparative radial artery pulse signal, and is further configured to obtain a first blood vessel ageing index based on the average value of the standard pulse area and the maximum value of the comparative pulse area determined thereby.

Another object of the present invention is to provide a blood vessel ageing index measuring and analyzing method.

Accordingly, a blood vessel ageing index measuring and analyzing method of the present invention comprises the steps of:

a) while a subject is in a relaxed state, measuring a pulse of the subject to obtain a standard radial artery pulse signal;

b) after obtaining the standard radial artery pulse signal, applying external pressure to a limb of the subject, followed by releasing the limb of the subject from the external pressure;

c) after releasing the limb of the subject from the external pressure, measuring a pulse of the subject to obtain a comparative radial artery pulse signal;

d) performing filter processing upon the standard radial artery pulse signal and the comparative radial artery pulse signal to obtain processed standard and comparative radial artery pulse signals;

e) digitizing the processed standard and comparative radial artery pulse signals to generate a digital standard radial artery pulse signal and a digital comparative radial artery pulse signal;

f) determining an average value of a standard pulse area with reference to the digital standard radial artery pulse signal;

g) determining a maximum value of a comparative pulse area with reference to the digital comparative radial artery pulse signal; and

h) obtaining a first blood vessel ageing index based on the average value of the standard pulse area and the maximum value of the comparative pulse area determined in steps f) and g).

### BRIEF DESCRIPTION OF THE DRAWINGS

Other features and advantages of the present invention will become apparent in the following detailed description of the preferred embodiment with reference to the accompanying drawings, of which:

FIG. 1 is a system block diagram of a preferred embodiment of a blood vessel ageing index measuring and analyzing system according to the present invention;

FIG. 2 is a schematic view illustrating an implementation of a pressure applying unit and a measuring unit of the blood vessel ageing index measuring and analyzing system;

FIG. 3 is a schematic view illustrating another implementation of the pressure applying unit;

FIG. 4 is a flow chart illustrating a preferred embodiment of a blood vessel ageing index measuring and analyzing method according to the present invention;

FIG. 5 shows a processed standard radial artery pulse signal obtained by measuring a pulse of a subject when the subject is in a relaxed state;

FIG. 6 shows a processed comparative radial artery pulse signal obtained by measuring a pulse of the subject after releasing a limb of the subject from an external pressure;

FIG. 7 is a flow chart showing steps performed by a blood vessel ageing index computing module of the blood vessel ageing index measuring and analyzing system; and

FIG. 8 is a graph illustrating dilatation of blood vessels due to action of endothelial cells.

### DETAILED DESCRIPTION OF THE PREFERRED EMBODIMENT

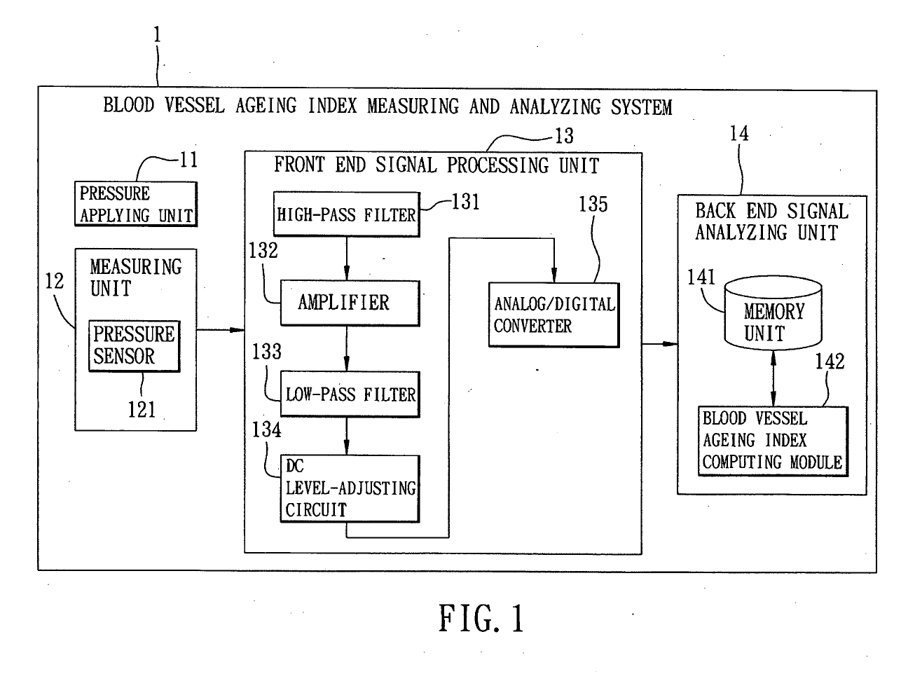

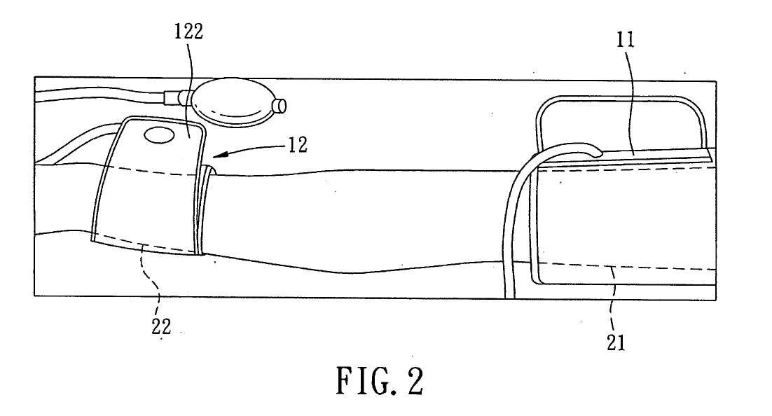

Referring to FIGS. 1 and 2, the preferred embodiment of a blood vessel ageing index measuring and analyzing system 1 according to the present invention includes a pressure applying unit 11, a measuring unit 12, a front end signal processing unit 13 coupled to the measuring unit 12, and a back end signal analyzing unit 14 coupled to the front end signal processing unit 13.

The pressure applying unit 11 is operable in a pressure-applying mode, where the pressure applying unit 11 is configured to apply external pressure to a limb 21 of a subject, or a pressure-releasing mode, where the pressure applying unit 11 is configured to release the limb 21 of the subject from the external pressure. In this embodiment, the pressure applying unit 11 is a pressure cuff and is configured to apply the external pressure, that is larger than a systolic pressure of the subject, to the limb 21 (e.g., an upper arm) of the subject when operated in the pressure-applying mode, such that corresponding humeral arteries of the subject are completely blocked. A pressure relief valve (not shown) of the pressure applying unit 11 is operated in the pressure-releasing mode for releasing the limb 21 of the subject from the external pressure. In this embodiment, the external pressure is a sum of the systolic pressure of the subject and a predetermined pressure (e.g., 40 mmHg).

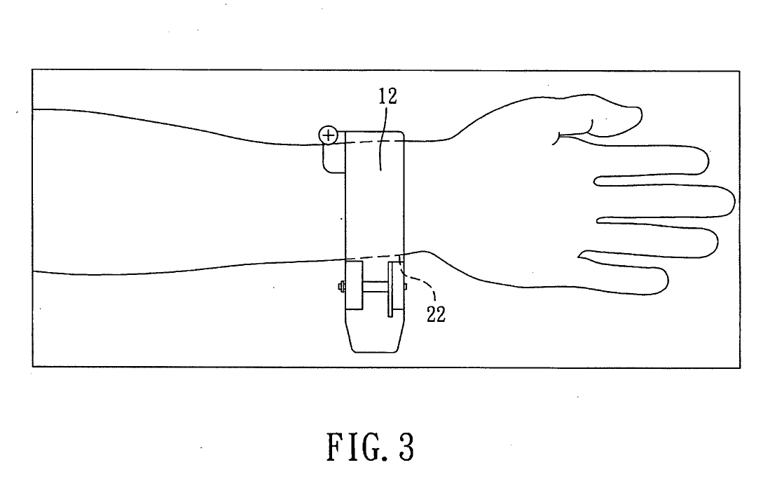

The measuring unit 12 is associated operatively with the pressure applying unit 11 for measuring pulses of the subject. The measuring unit 12 is configured to output a standard radial artery pulse signal (i.e., a baseline pulse signal) based upon a pulse of the subject that was measured prior to operation of the pressure applying unit 11 in the pressure-applying mode. The measuring unit 12 is further configured to output a comparative radial artery pulse signal (i.e., an RH pulse signal) based upon a pulse of the subject that was measured after operation of the pressure applying unit 11 in the pressure-releasing mode. The measuring unit 12 includes a pressure sensor 121 configured to detect a radial artery pulse at a wrist 22 of the subject. In this embodiment, the pressure sensor 121 is a resistive-type pressure sensor. The measuring unit 12 further includes a pressure cuff 122 that is in fluid communication with the pressure sensor 121, i.e., the resistive-type pressure sensor. The pressure cuff 122 is configured to be disposed at the wrist 22, and to apply a constant pressure (e.g., 40 mmHg) for measuring variations in blood vessel diameter via the fluid communication between the pressure sensor 121 and the pressure cuff 122. It should be noted that, as shown in FIG. 3, the measuring unit 12 may be implemented as a wrist-clamping-type detecting device that is configured to apply the constant pressure. The pressure sensor 121 of the wrist-clamping-type detecting device is a skin-contact-type pressure sensor that is configured to contact the wrist 22 of the subject for directly measuring the radial artery pulse. The measuring unit 12 is not limited to the implementations disclosed herein.

The front end signal processing unit 13 is configured to perform filter processing upon the standard and comparative radial artery pulse signals outputted by the measuring unit 12 to obtain processed standard and comparative radial artery pulse signals, and is further configured to digitize the processed standard and comparative radial artery pulse signals to generate a digital standard radial artery pulse signal and a digital comparative radial artery pulse signal. The front end signal processing unit 13 includes a high-pass filter 131, an amplifier 132, a low-pass filter 133, a DC level-adjusting circuit 134, and an analog/digital converter 135. The high-pass filter 131, amplifier 132, low-pass filter 133, and DC level-adjusting circuit 134 are configured to perform the filter processing, and the analog/digital converter 135 is configured to perform the digitizing. In this embodiment, the high-pass filter 131 is a second-order high-pass filter, the amplifier 132 is a non-inverting amplifier, and the low-pass filter 133 is a second-order low-pass filter. Since the operations of each of the above-mentioned components of the front end signal processing unit 13 are well known to those skilled in the art, further details thereof will be omitted herein for the sake of brevity.

The back end signal analyzing unit 14 is configured to obtain a first blood vessel ageing index and a second blood vessel ageing index according to the digital standard and comparative radial artery pulse signals outputted by the front end signal processing unit 13. The back end signal analyzing unit 14 includes a memory unit 141 and a blood vessel ageing index computing module 142. The digital standard and comparative radial artery pulse signals are stored in the memory unit 141. In practice, the blood vessel ageing index computing module 142 may be realized through proprietary computer software for computing the first and second blood vessel ageing indices. In this embodiment, the back end signal analyzing unit 14 may be a computer loaded with the blood vessel ageing index computing module 142.

The preferred embodiment of a blood vessel ageing index measuring and analyzing method performed using the system 1 will now be described in the succeeding paragraphs.

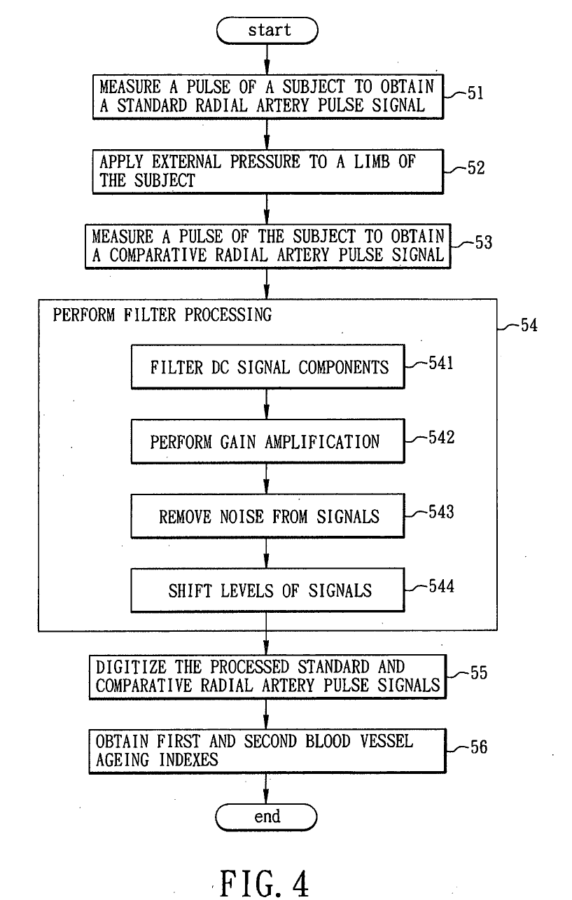

Referring to FIGS. 1, 2 and 4, before the pressure applying unit 11 applies an external pressure to the subject, the subject is in a relaxed state for a duration of time (such as 4 minutes). At this time, the measuring unit 12 measures a pulse of the subject to obtain a standard radial artery pulse signal in step 51.

In step 52, the pressure applying unit 11 is operated in the pressure-applying mode to apply the external pressure to the limb 21 (i.e., upper arm) of the subject for a duration of time (such as 2 minutes). At this time, since the external pressure is larger than the systolic pressure of the subject, corresponding humeral arteries of the subject are completely blocked, such that blood cannot flow to the forearm of the subject. During this duration, endothelial cells are stimulated to liberate nitric-oxide (NO) for controlling dilatation of blood vessels. Subsequently, the pressure applying unit 11 is operated in the pressure-releasing mode to release the limb 21 from the external pressure. After releasing the limb 21 of the subject from the external pressure, the measuring unit 12 measures a pulse of the subject to obtain a comparative radial artery pulse signal in step 53.

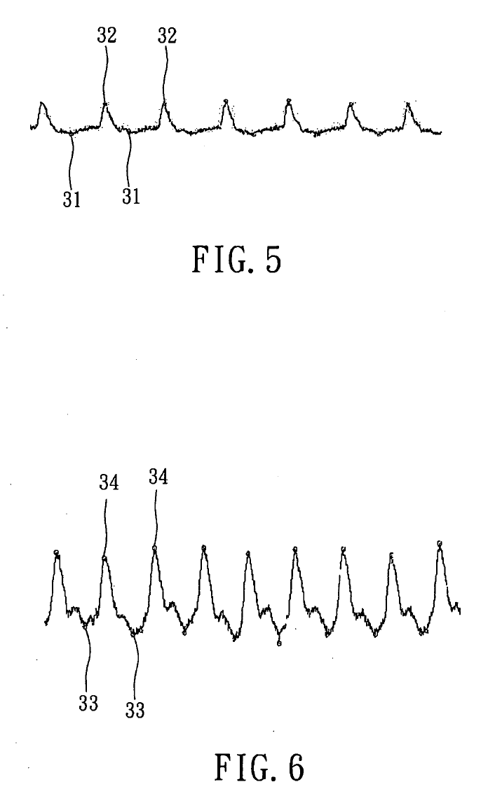

Then, in step 54, the high-pass filter 131, amplifier 132, low-pass filter 133, and DC level-adjusting circuit 134 of the front end signal processing unit 13 operate to perform filter processing that includes the following sub-steps. The high-pass filter 131 is operable so as to filter DC signal components from the standard and comparative radial artery pulse signals in sub-step 541, followed by operation of the amplifier 132 in sub-step 542 for amplification. Then, the low-pass filter 133 is operable so as to remove noise attributed to the pressure applying unit 11 and the measuring unit 12 rubbing against the skin of the subject, and to surrounding 60-Hz interference noise from the amplified standard and comparative radial artery pulse signals in sub-step 543 to obtain robust standard and comparative radial artery pulse signals. Finally, in sub-step 544, the DC level-adjusting circuit 134 is operable so as to shift levels of the robust standard and comparative radial artery pulse signals to a predetermined level (e.g., larger than 0V) to obtain processed standard and comparative radial artery pulse signals (see FIGS. 5 and 6).

In step 55, the analog/digital converter 135 of the front end signal processing unit 13 is operable so as to digitize the processed standard and comparative radial artery pulse signals obtained in sub-step 544 to generate a digital standard radial artery pulse signal and a digital comparative radial artery pulse signal.

Finally, in step 56, the back end signal analyzing unit 14 is operable so as to obtain a first blood vessel ageing index and a second blood vessel ageing index for evaluating the endothelial cell function and the blood vessel ageing.

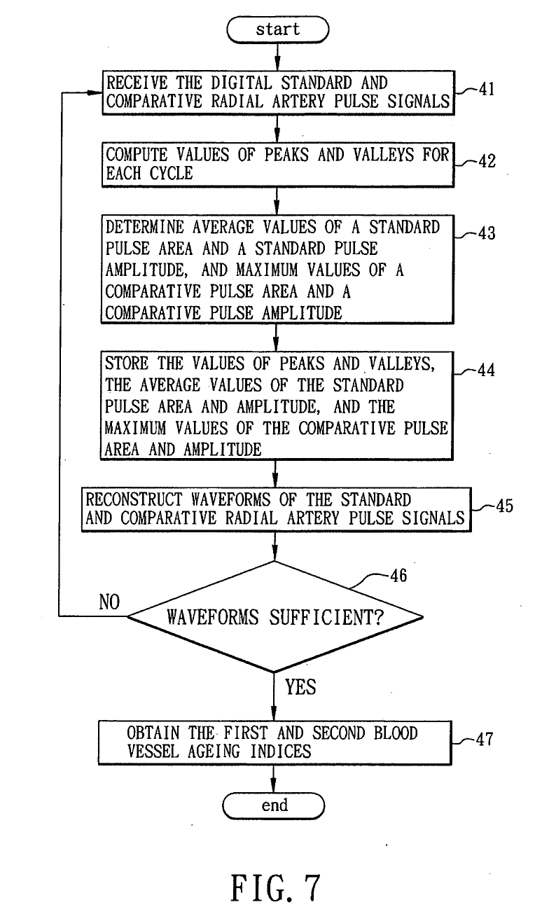

Referring to FIGS. 1 and 5 to 7, step 56 includes the following sub-steps. In sub-step 41, the back end signal analyzing unit 14 is operable so as to receive the digital standard and comparative radial artery pulse signals outputted by the front end signal processing unit 13, and to store the digital standard and comparative radial artery pulse signals in the memory unit 141.

In sub-step 42, the blood vessel ageing index computing module 142 is operable so as to compute a value of peak 32 and a value of valley 31 for each cycle of the digital standard radial artery pulse signal, and a value of peak 34 and a value of valley 33 for each cycle of the digital comparative radial artery pulse signal.

In sub-step 43, the blood vessel ageing index computing module 142 is operable so as to compute an area value and an amplitude value for each cycle of the digital standard radial artery pulse signal according to the value of peak 32 and the value of valley 31. The blood vessel ageing index computing module 142 is further operable so as to compute an area value and an amplitude value for each cycle of the digital comparative radial artery pulse signal according to the value of peak 34 and the value of valley 33. Then, the blood vessel ageing index computing module 142 determines an average value of a standard pulse area (A_{Standard}) with reference to the area values of the digital standard radial artery pulse signal, and a maximum value of a comparative pulse area (A_{Comparative}) with reference to the area values of the digital comparative radial artery pulse signal. The blood vessel ageing index computing module 142 further determines an average value of a standard pulse amplitude (Amp_{Standard}) with reference to the amplitude values of the digital standard radial artery pulse signal, and a maximum value of a comparative pulse amplitude (Amp_{Comparative}) with reference to the amplitude values of the digital comparative radial artery pulse signal.

In sub-step 44, the values of peaks 32, 34, the values of valleys 31, 33, the average value of the standard pulse area, the maximum value of the comparative pulse area, the average value of the standard pulse amplitude, and the maximum value of the comparative pulse amplitude are stored in the memory unit 142.

In sub-step 45, the blood vessel ageing index measuring and analyzing system 1 is operable so as to reconstruct waveforms of the digital standard and comparative radial artery pulse signals via a commercially available graphic software interface.

Sub-step 46 is to determine through the blood vessel ageing index computing module 142 whether the waveforms reconstructed in sub-step 45 are sufficient. If insufficient, the flow goes back to sub-step 41.

When the waveforms are sufficient, the first blood vessel ageing index (DI-Area) is obtained in sub-step 47 via the blood vessel ageing index computing module 142 based upon the average value of the standard pulse area (A_{Standard}) and the maximum value of the comparative pulse area (A_{Comparative}). Further, the second blood vessel ageing index (DI-Amplitude) is obtained in sub-step 47 via the blood vessel ageing index computing module 142 based upon the average value of the standard pulse amplitude (Amp_{Standard}) and the maximum value of the comparative pulse amplitude (Amp_{Comparative}). The first and second blood vessel ageing indices is computed based upon the following equations,

DI−Area=A_{Standard}/A_{Comparative} (2)

DI−Amplitude=Amp_{Standard}/Amp_{Comparative} (3)

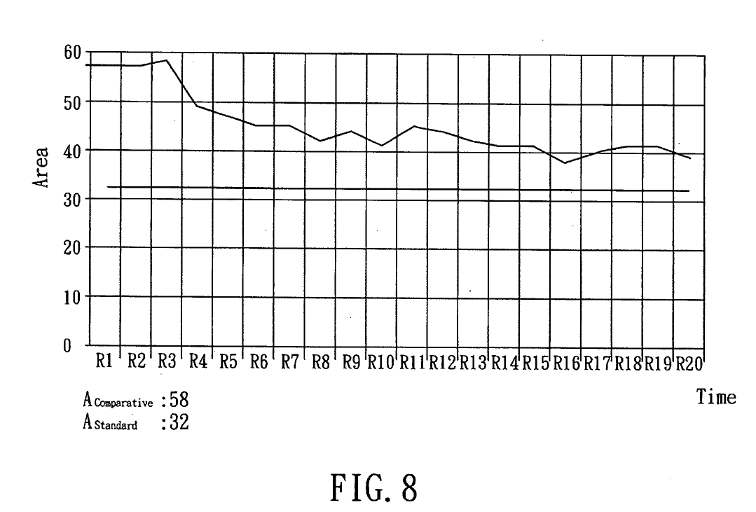

Referring to FIG. 8, as an example, the average value of the standard pulse area is 32 (i.e., A_{Standard}=32), and the maximum value of the comparative pulse area is 58 (i.e., A_{Comparative}=58). Accordingly, the first blood vessel ageing index is 1.8125 (i.e., DI-Area=1.8125).

According to medical documents, a value of internal pressure of a blood vessel is proportional to a vascular volume of the blood vessel. When the heart contracts, due to elasticity of the arterial walls, blood pressure in the artery increases with the outward dilatation of the wall of the blood vessel, such that the amount of blood outputted from the heart increases. When the heart relaxes, the blood pressure decreases with the restoration of the wall of the blood vessel, such that the amount of blood outputted from the heart decreases. Therefore, the wall of the blood vessel periodically pulsates with heartbeats. With variation of the pressure in the artery, the flow of blood pulsates with this variation. Due to the period of systole and diastole, the variation of the pressure in the artery causes a variation in the vascular volume, and the amount of the variation in the vascular volume is proportional to the amount of variation of the pressure in the artery. The system of the present invention is used to measure the radial artery pulse at the wrist 22 (shown in FIG. 2) of the subject, and directly senses the variation of the blood vessel diameter due to nitric oxide for obtaining an index suitable for evaluating health of blood vessels and an early stage of atherosclerosis.

As mentioned hereinabove, the system of the present invention determines the first and second blood vessel ageing indices for evaluating the endothelial cell function of the subject. A higher index indicates healthier blood vessels. The endothelial cell function is repairable, and therefore, a user can detect the health conditions of the blood vessels per week or per month himself/herself for achieving a purpose of tracking the health of blood vessels in the long term. When an index indicates that the endothelial cell function is poor, the user can adjust his/her routine living habits for improving the endothelial cell function and for alleviating ageing of the blood vessels.

In sum, compared with U.S. Pat. No. 7,077,809, the blood vessel ageing index measuring and analyzing system 1 according to the present invention has the following advantages.

First, the system of U.S. Pat. No. 7,077,809 measures capillaries of digits that are the smallest branch of arteries and therefore, captures weaker signals. On the other hand, the system 1 of the present invention measures the radial artery pulse at the wrist 22 and captures stronger signals according to the variation of the arterial walls.

Second, the system of U.S. Pat. No. 7,077,809 includes a digit-clamping device that includes infrared light emitting and receiving units, and that needs a specific mold for manufacture. Therefore, the cost of the system is relatively high, and maintenance of the system is uneasy. Moreover, angular drift of the infrared light emitting and receiving units can result in errors. On the other hand, the pressure sensor 121 (a pressure cuff or a wrist-clamping-type detecting device) of the system 1 of the present invention is commercially available, and is easy to replace. Further, there is no such problem that the pressure sensor 121 is disposed inappropriately.

Third, the system of U.S. Pat. No. 7,077,809 measures the condition of the flow of blood through the infrared light emitting and receiving units for determining elasticity of blood vessels. This is indirect measurement. However, by using the pressure sensor 121 to determine the elasticity of blood vessels, the system 1 of the present invention directly measures the variation of the blood vessel diameter caused by nitric oxide.

While the present invention has been described in connection with what is considered the most practical and preferred embodiment, it is understood that this invention is not limited to the disclosed embodiment but is intended to cover various arrangements included within the spirit and scope of the broadest interpretation so as to encompass all such modifications and equivalent arrangements.

What is claimed is:

1. A blood vessel ageing index measuring and analyzing system, comprising:
a pressure applying unit operable in one of a pressure-applying mode, where said pressure applying unit is configured to apply external pressure to a limb of a subject, and a pressure-releasing mode, where said pressure applying unit is configured to release the limb of the subject from the external pressure;
a measuring unit associated operatively with said pressure applying unit and configured to output a standard radial artery pulse signal based upon a pulse of the subject that was measured prior to operation of said pressure applying unit in the pressure-applying mode, and to output a comparative radial artery pulse signal based upon a pulse of the subject that was measured after operation of said pressure applying unit in the pressure-releasing mode;
a front end signal processing unit coupled to said measuring unit, configured to perform filter processing upon the standard radial artery pulse signal and the comparative radial artery pulse signal outputted by said measuring unit to obtain processed standard and comparative radial artery pulse signals, and further configured to digitize the processed standard and comparative radial artery pulse signals to generate a digital standard radial artery pulse signal and a digital comparative radial artery pulse signal; and
a back end signal analyzing unit coupled to said front end signal processing unit, configured so as to determine an average value of a standard pulse area with reference to the digital standard radial artery pulse signal, and so as to determine a maximum value of a comparative pulse area with reference to the digital comparative radial artery pulse signal, and further configured to obtain a first blood vessel ageing index based on the average value of the standard pulse area and the maximum value of the comparative pulse area determined thereby.

2. The blood vessel ageing index measuring and analyzing system as claimed in claim 1, wherein said measuring unit includes a pressure sensor configured to detect a radial artery pulse at a wrist of the subject.

3. The blood vessel ageing index measuring and analyzing system as claimed in claim 2, wherein said pressure sensor is a resistive-type pressure sensor.

4. The blood vessel ageing index measuring and analyzing system as claimed in claim 3, wherein said measuring unit further includes a pressure cuff that is in fluid communication with said pressure sensor.

5. The blood vessel ageing index measuring and analyzing system as claimed in claim 2, wherein said pressure sensor is a skin-contact-type pressure sensor.

6. The blood vessel ageing index measuring and analyzing system as claimed in claim 1, wherein said pressure applying unit is a pressure cuff and is configured to apply the external pressure, that is larger than a systolic pressure of the subject, to an upper arm of the subject when operated in the pressure-applying mode.

7. The blood vessel ageing index measuring and analyzing system as claimed in claim 1, wherein the first blood vessel ageing index is calculated by said back end signal analyzing unit as a quotient of the maximum value of the comparative pulse area divided by the average area of the standard pulse area.

8. The blood vessel ageing index measuring and analyzing system as claimed in claim 1, wherein said back end signal analyzing unit is further configured so as to determine an average value of a standard pulse amplitude with reference to the digital standard radial artery pulse signal, and so as to determine a maximum value of a comparative pulse amplitude with reference to the digital comparative radial artery pulse signal, and is further configured to obtain a second blood vessel ageing index based on the average value of the standard pulse amplitude and the maximum value of the comparative pulse amplitude determined thereby.

9. The blood vessel ageing index measuring and analyzing system as claimed in claim 8, wherein the first blood vessel ageing index is calculated by said back end signal analyzing unit as a quotient of the maximum value of the comparative pulse area divided by the average area of the standard pulse area.

10. The blood vessel ageing index measuring and analyzing system as claimed in claim 9, wherein the second blood vessel ageing index is calculated by said back end signal analyzing unit as a quotient of the maximum value of the comparative pulse amplitude divided by the average value of the standard pulse amplitude.

11. The blood vessel ageing index measuring and analyzing system as claimed in claim 8, wherein the second blood vessel ageing index is calculated by said back end signal analyzing unit as a quotient of the maximum value of the comparative pulse amplitude divided by the average value of the standard pulse amplitude.

12. A blood vessel ageing index measuring and analyzing method, comprising the steps of:
a) while a subject is in a relaxed state, measuring a pulse of the subject to obtain a standard radial artery pulse signal;
b) after obtaining the standard radial artery pulse signal, applying external pressure to a limb of the subject, followed by releasing the limb of the subject from the external pressure;
c) after releasing the limb of the subject from the external pressure, measuring a pulse of the subject to obtain a comparative radial artery pulse signal;
d) performing filter processing upon the standard radial artery pulse signal and the comparative radial artery pulse signal to obtain processed standard and comparative radial artery pulse signals;
e) digitizing the processed standard and comparative radial artery pulse signals to generate a digital standard radial artery pulse signal and a digital comparative radial artery pulse signal;
f) determining an average value of a standard pulse area with reference to the digital standard radial artery pulse signal;
g) determining a maximum value of a comparative pulse area with reference to the digital comparative radial artery pulse signal; and
h) obtaining a first blood vessel ageing index based on the average value of the standard pulse area and the maximum value of the comparative pulse area determined in steps f) and g).

13. The blood vessel ageing index measuring and analyzing method as claimed in claim 12, wherein, in steps a) and c), a pressure sensor is used to detect a radial artery pulse at a wrist of the subject.

14. The blood vessel ageing index measuring and analyzing method as claimed in claim 12, wherein, in step b), the external pressure is applied to an upper arm of the subject and is larger than a systolic pressure of the subject.

15. The blood vessel ageing index measuring and analyzing method as claimed in claim 12, wherein step d) includes the following sub-steps:
d1) filtering direct current signal components from the standard and comparative radial artery pulse signals;
d2) amplifying the filtered standard and comparative radial artery pulse signals obtained in sub-step d1); and
d3) removing noise from the amplified standard and comparative radial artery pulse signals obtained in sub-step d2) to obtain the processed standard and comparative radial artery pulse signals.

16. The blood vessel ageing index measuring and analyzing method as claimed in claim 12, wherein the first blood vessel ageing index is calculated as a quotient of the maximum value of the comparative pulse area divided by the average area of the standard pulse area.

17. The blood vessel ageing index measuring and analyzing method as claimed in claim 12, further comprising the steps of:
i) determining an average value of a standard pulse amplitude with reference to the digital standard radial artery pulse signal;
j) determining a maximum value of a comparative pulse amplitude with reference to the digital comparative radial artery pulse signal; and
k) obtaining a second blood vessel ageing index based on the average value of the standard pulse amplitude and the maximum value of the comparative pulse amplitude determined in steps i) and j).

18. The blood vessel ageing index measuring and analyzing method as claimed in claim 17, wherein the first blood vessel ageing index is calculated as a quotient of the maximum value of the comparative pulse area divided by the average area of the standard pulse area.

19. The blood vessel ageing index measuring and analyzing method as claimed in claim 18, wherein the second blood vessel ageing index is calculated as a quotient of the maximum value of the comparative pulse amplitude divided by the average value of the standard pulse amplitude.

20. The blood vessel ageing index measuring and analyzing method as claimed in claim 17, wherein the second blood vessel ageing index is calculated as a quotient of the maximum value of the comparative pulse amplitude divided by the average value of the standard pulse amplitude.
